(19) United States
(12) Patent Application Publication
(10) Pub. No.: US 2004/0143310 A1
(43) Pub. Date: Jul. 22, 2004

(54) Burn heat therapy instrument

(76) Inventor: Tzu-Chen Cheng, Mirador Norte (DO)

Correspondence Address: Tzu-Chen Cheng, Av. 27 de Febrero, #418, Mirador Norte., La Feria, P.O. Box 165-2, Santo Domingo, D.N., DO

(21) Appl. No.: 10/347,101

(22) Filed: Jan. 17, 2003

(51) Int. Cl. A61F 7/00; A61F 7/12

(52) U.S. Cl. 607/96

(57) ABSTRACT

A burn heat therapy instrument has a handle tube 1. This handle tube has in its inferior part horizontal or round holes, called cut heat expansion holes 2, around it. The handle tube 1 is joined in its inferior extreme to a burned bronze heating tube 3. Inside of the burned bronze heating tube 3, there is a metal spring 5, with a round metal 9 in its lower part. The burned bronze heating tube 3 is joined to a metal ashtray 8.

### BACKGROUND OF THE INVENTION

1. Field of the Invention

This invention relates generally to heat therapy, including heat therapy technique and instrument for its application. This invention is classified as a utility invention according to the United State Patent Classification.

2. Prior Art.

Historically, heat has been a natural remedy used both to relieve pain and enhance the recovery process. By increasing the temperature of the skin surface and underlying tissues, heat stimulates the thermoreceptors—sensory receptors that respond to heat and cold—which then help block transmissions of pain signals to the brain. This translates into a significant decrease of discomfort. Heat therapy increases blood flow, which helps to decrease stiffness, relaxes sore muscles, and provides soothing comfort. Many thermotherapy techniques and instrument are based on hot water and hot air as heating medium.

### SUMMARY OF THE INVENTION

This invention provides a technique and apparatus based on heat therapy, which are more convenient, secure and efficient, because of its structure and heating medium.

### DRAWINGS—FIGURES

FIG. 1 illustrates the physical form of the instrument.

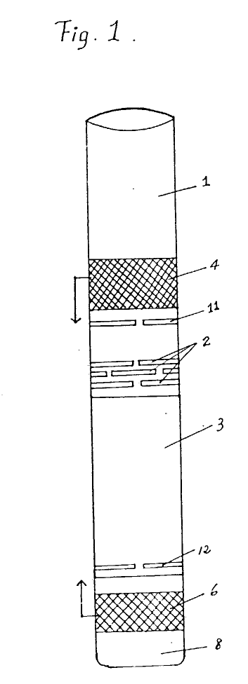

FIG. 2 shows the metal spring included in the instrument.

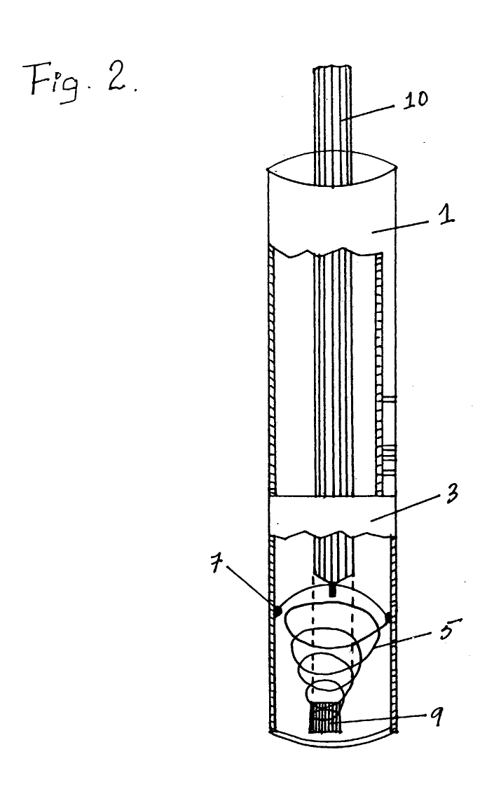

### DRAWINGS—REFERENCE NUMERALS

1 Handle tube 7 Two screws

2 cut heat expansion holes 8 metal ashtray

3 bronze heating tube 9 round metal

4 vitrified fibre ring 10 sandal wood incense

5 metal spring 11 air holes

6 vitrified fibre ring 12 air holes

### DESCRIPTION OF THE INVENTION

The Technique

One can manage easily the instrument oneself. To handle the instrument, one take it by the handle tube 1, which is cold and safe to handle, because, even the handle tube 1 is joined in its inferior extreme to the burned bronze heating tube 3, but the heat can't expand to the handle tube 1. One should cover the skin area with a piece of cloth to avoid direct contact with the burned bronze heating tube 3, which is hot. The person using the instrument can control the temperature applicated to the skin by moving the heating tube 3 in al directions, by pressing or relieving the heating tube against the skin, or staying in the same surface area to increase to the desired and tolerable temperature.

The invention has two applications:

1) Firstly, one can use the instrument to enhance body blood flow, by massaging the heating tube 3 in the internal side of the arms and the internal side of the legs, where we can find the main or principal blood vessels to the arms and thighs.

2) The other application consists in relieving muscular spasm, resolution of inflammatory infiltrated, pain relief, by applicating heating tube 3 to the skin of the problem area.

The Invention

The burn heat therapy instrument has a handle tube 1, made of stainless. This handle tube is to be handle by one to operate the instrument and to dispose the sandal wood incense 10. This handle tube has in its inferior part horizontal or round holes, called cut heat expansion holes 2, around it. The function of said cut heat expansion holes are to avoid the heat expansion from burned bronze heating tube 3 to the handle tube 1. This is a technique invented by me to cut the heat expansion in a metal material body. Above the cut heat expansion holes 2, there are horizontal holes, called air holes 11, to help smoke escape. The handle tube 1 is joined in its inferior extreme to a burned bronze heating tube 3. This burned bronze heating tube is a medium to transfer the heat from the instrument to the skin by indirect contact through a piece of cloth to prevent skin injury. Said burned bronze heating tube 3 has air holes 12, for oxygenate the sandal wood incense 10 in its burning. Inside of the burned bronze heating tube 3, there is a metal spring 5, with a round metal 9 in its lower part. The burning extreme of the sandal wood incense 10 makes contact with the metal spring 5 and the round metal 9, and transfers the transfer the heat to them and the burned bronze heating tube 3. Two screws 7 suspend said metal spring 5 inside the burned bronze heating tube. The burned bronze heating tube 3 is joined to a metal ashtray 8. The ashes from the burning sandal wood incense will deposit in the metal ashtray 8. The metal ashtray can be removed to clean the ashes. A vitrified fibre ring 4 can cover the cut heat expansion holes 2 in the handle tube 1 to conserve and to grade heat level. Also, a vitrified fibre ring 6 can cover the air holes 12 in the burned bronze heating tube to extinguish the sandal wood incense burning.

I claim:

1. An instrument created to therapeutically treat a problem in the body of a patient in a limited skin area and to promote good health, said instrument comprising:
A. Means to generate heat temperature by burning.
B. Means to operate the instrument and transfer said heat temperature to located skin area in the body.

2. An instrument as set forth in claim 1, wherein burning sandal wood incense generates the heat in the burned bronze heating tube.

3. An instrument as set forth in claim 2, wherein the metal spring is positioned to separate ashes from the burning sandal wood incense, and help the sandal wood incense keeps burning.

4. An instrument as set forth in claim 3, wherein the horizontal or round air holes in the burning bronze heating tube are made to let air circulation and oxygenate the burning sandal wood incense.

5. An instrument as set forth in claim 3, wherein the ashes from the burning sandal wood incense are deposit in a metal ashtray.

6. An instrument as set forth in claim 3, wherein two screws inside the burning bronze heating tube suspend the metal spring.

7. An instrument as set forth in claim 2, wherein the burned bronze heating tube is used as a medium to transfer the heat to skin area.

8. An instrument as set forth in claim 1, wherein a vitrified fibre ring can cover the air holes in the burned bronze heating tube to extinguish the sandal wood incense burning.

9. An instrument as set forth in claim 1, wherein a vitrified fibre ring can cover the cut heat expansion holes in the handle tube to conserve and to grade heat level.
